(19) United States
(12) Patent Application Publication
(10) Pub. No.: US 2026/0179754 A1
(43) Pub. Date: Jun. 25, 2026

(54) Global Resonance App: Synchronization of Frequencies for Meditation and Global Connectivity

(71) Applicant: Alexander Thompson-Westfall, Vicksburg, MI (US)

(72) Inventor: Alexander Thompson-Westfall, Vicksburg, MI (US)

(21) Appl. No.: 18/991,586

(22) Filed: Dec. 22, 2024

(51) Int. Cl. G16H 20/70; G16H 80/00

(52) U.S. Cl. CPC G16H 20/70; G16H 80/00

(57) ABSTRACT

The Global Resonance App enables synchronized meditation by aggregating and harmonizing frequencies across multiple users globally. Users select individual frequencies that dynamically contribute to a collective frequency pool, creating immersive visual and auditory experiences enhanced by optional biofeedback integration.

### BACKGROUND OF THE INVENTION

The rapid growth of meditation and mindfulness applications has demonstrated a significant need for tools that promote mental well-being, focus, and relaxation. Despite the proliferation of such applications, most focus on isolated user experiences and lack features that create shared or community-driven meditative environments. Current meditation apps often feature basic soundscapes or guided sessions that do not account for collective user engagement or real-time synchronization across users globally. Additionally, most fail to integrate biofeedback devices, missing an opportunity to personalize meditation experiences based on physiological data. The Global Resonance App seeks to address these limitations by introducing frequency synchronization, allowing users across the globe to experience meditation collectively. Through harmonic resonance, users not only meditate individually but also participate in a synchronized network that enhances collective well-being.

### SUMMARY OF THE INVENTION

The Global Resonance App is an innovative platform designed to synchronize frequencies across multiple users. This app aggregates individual user frequencies to compute a collective global frequency. Through binaural beats, harmonic overlays, and visual representations, users experience immersive and synchronized meditation. The app's core functionalities include real-time user contributions to a global frequency pool, dynamic visualization of frequencies, and optional biofeedback integration.

### DETAILED DESCRIPTION

1. User Device Interface: Users access the app through mobile devices or tablets. The app's intuitive interface allows them to select personal frequencies, adjust soundscapes, and visualize harmonic pulsations. The interface features customizable presets such as Schumann resonances, Solfeggio frequencies, and other meditative tones. 2. Cloud-Based Frequency Aggregator: User inputs are transmitted to a centralized cloud server. The aggregator computes the average of all user-selected frequencies, creating a dynamically updating global frequency. 3. Visualization Engine: A visual pulsating globe or waveform representation reflects the real-time frequency alignment between the user's input and the global average. Visual elements adjust based on intensity, amplitude, and frequency shifts. 4. Biofeedback Integration: Optional biofeedback devices (e.g., heart rate monitors, EEG sensors) are supported. The app modifies output frequencies to align with physiological data, offering a personalized experience tailored to the user's mental and physical state. 5. Group Meditation Feature: Users can participate in synchronized group meditation sessions, either globally or within smaller peer-to-peer groups. A shared frequency creates collective resonance, enhancing the overall experience.

1. A frequency synchronization system comprising: A mobile application interface for user frequency selection; A cloud-based aggregator computing global average frequencies; A visualization engine displaying pulsating harmonic graphics; An optional biofeedback integration module for real-time adjustment of frequencies.

2. The system of claim 1, wherein the app generates binaural beats based on user-defined and globally averaged frequencies.

3. The system of claim 1, wherein peer-to-peer connectivity facilitates group meditation with shared synchronized frequencies.

4. The system of claim 1, further including presets for known resonance frequencies, such as Schumann and Solfeggio frequencies.
